(19) United States
(12) Patent Application Publication
(10) Pub. No.: US 2004/0156888 A1
(43) Pub. Date: Aug. 12, 2004

(54) Liposomal formulations

(76) Inventors: Gerard M. Jensen, Brea, CA (US); Ning Hu, San Gabriel, CA (US); Su-Ming Chiang, West Hills, CA (US); Craig Skenes, La Mirada, CA (US); Richard Fahrner, Tustin, CA (US)

Correspondence Address: Schwegman, Lundberg, Woessner & Kluth, P.A., P.O. Box 2938, Minneapolis, MN, 55402, US

(21) Appl. No.: 10/723,423

(22) Filed: Nov. 26, 2003

Related U.S. Application Data
(60) Provisional application No. 60/429,122, filed on Nov. 26, 2002.

(51) Int. Cl. A61K 38/14; A61K 48/00; A61K 9/127; C12N 15/88

(52) U.S. Cl. 424/450; 424/649; 514/8; 514/44; 435/458

(57) ABSTRACT

The invention provides a formulation comprising a lipophobic therapeutic agent encapsulated in a liposome having improved efficacy and/or reduced toxicity.

### PRIORITY OF INVENTION

This application claims priority from U.S. Provisional Application No. 60/429,122, filed 26 Nov. 2002.

### BACKGROUND OF THE INVENTION

Liposomes are sub-micron spherical vesicles comprised of phospholipids and cholesterol that form a hydrophobic bilayer surrounding an aqueous core. These structures have been used with a wide variety of therapeutic agents and allow for a drug to be entrapped within the liposome based in part upon its own hydrophobic (bilayer entrapment) or hydrophilic properties (entrapment in the aqueous compartment).

Typically, encapsulating a drug in a liposome can alter the pattern of biodistribution and the pharmacokinetics for the drugs. In certain cases, liposomal encapsulation has been found to lower the toxicity. In particular, so-called, long circulating liposomal formulations, which avoid uptake by the organs of the mononuclear phagocyte system, primarily in the liver and spleen, have been extensively studied. Such long-circulating liposomes may include a surface coat of flexible water soluble polymer chains that act to prevent interaction between the liposome and plasma components that play a role in liposome uptake, or such liposomes can be made without this coating but of saturated, long-chain phospholipids and cholesterol.

Cisplatin has been widely used for over thirty years in treating numerous solid tumors and continues to play an essential role in the treatment of cancer. Although the compound is an effective anti-tumor agent, its use has been limited due to its severe cumulative renal toxicity, neurotoxicity, myelosuppression, and ototoxicity.

The pharmacokinetics, tissue distribution, and therapeutic effectiveness of cisplatin in long-circulating (e.g. pegylated) liposomes (SPI-077) has been investigated: see for example, M. S. Newman et al., Cancer Chemother Pharmacol, 1999, 43, 524; S. Bandak et al., Anti-Cancer Drugs, 1999, 10, 911-920; M. D. DeMario et al., Proceedings of ASCO, 1998, 17, 883; P. K. Working et al., Toxicological Sciences, 1998, 46, 155-165; J. M. Terwogt et al., Cancer Chemother Pharmacol, 2002, 49, 201-210; C. T. Colbern et al., Journal of Inorganic Biochemistry, 1999, 77, 117-120 and G. J. Veal et al., British Journal of Cancer, 2001, 84, 1029-1035. The anti-tumor activity of doxorubicin in Peg-coated liposomes has also been investigated by R-L Hong, Clinical Cancer Research, 1999, 5, 3645-3652.

Alza (now Johnson & Johnson) developed SPI-077, a liposomal cisplatin, through Phase I-II clinical trials. The SPI-077 candidate was formulated into a PEG-coated long circulating liposome yielding minimal release of free drug from the liposome, while avoiding the renal clearance mechanisms common for the free drug. The side effect profile of SPI-077 was significantly better than that of the free drug, however SPI-077 was also found to have lower efficacy in limited human testing and further development of that liposomal formulation has apparently been abandoned.

Although encapsulation in long-circulating pegylated liposomes has been found to lower the toxicity of certain specific therapeutic agents, such encapsulation has not been found to be generally useful for improving the effectiveness of a broad group of therapeutic agents. For example, in one report, cisplatin encapsulated in pegylated liposomes was found to be essentially inactive against squamous cancers of the head and neck. See K. J. Harrington et al., Anals of Oncology, 2001, 12, 493-496. This lack of general success results from an inability to properly balance the enhanced circulation lifetime of the liposomes with specific drug release profiles. Thus, although investigators have successfully increased the circulation lifetimes of drugs encapsulated in pegylated liposomes, which benefically promotes accumulation of the liposomes at tumor growth sites, they have been unable to realize acceptable drug release profiles from these liposomes for certain therapeutic agents. Accordingly, drugs encapsulated in such pegylated liposomes typically have been found to demonstrate similar or diminished clinical activity compared to the corresponding non-encapsulated drugs.

H. J. Lim et al., The Journal of Pharmacology and Experimental Therapeutics, 1997, 281, 566-573 investigated the balance between liposome delivery to a disease site and drug release for a liposomal (DMPC/cholesterol) encapsulated formulation of the amphiphilic and gradient loadable antineoplastic agent mitoxantrone. This liposomal formulation was found to improve the antitumor activity of the compound in a BDF 1 mouse model. The anti-tumor effects of mitoxantrone in programmable fusogenic vesicles was also investigated by G. Adlakha-Hutcheon et al., Nature Biotechnology, 1999, 17, 775-779. Improved anti-tumor activity was reported compared to three other liposomal formulations.

In spite of the extensive research that has been carried out on long-circulating pegylated liposomes, there remains a need for liposomal formulations that are generally useful for improving the therapeutic index and the activity of therapeutic agents. Although improvements in antitumor activity have been reported for certain specific liposomal formulations of the amphiphilic agent mitoxantrone, no liposomal system has been identified that is generally useful for improving the therapeutic index and the activity of non-amphiphilic therapeutic agents.

### SUMMARY OF THE INVENTION

Applicant has discovered that beneficial therapeutic effects can be achieved by encapsulating a lipophobic therapeutic agent in a liposome that increases the elimination half-life of the agent to a value that is at least as great as the value of the free drug but less than values typically achieved by long-circulating (e.g. pegylated liposomes). Such liposomal systems are useful for improving the therapeutic index and/or the activity of lipophobic therapeutic agents. Accordingly, in one embodiment, the invention provides a formulation comprising a lipophobic therapeutic agent encapsulated in a liposome, wherein, 1) the elimination half-life of the therapeutic agent when administered to an animal as part of the formulation is at least as long as the elimination half-life of the therapeutic agent when administered to the same animal in the absence of the liposome, and wherein 2) the elimination half-life of the therapeutic agent when administered as part of the formulation is less than about 14 hours in a rat. Liposomal formulations with the same elimination half life as the free drug may still afford beneficial alteration in tissue distribution or reduction in volume of distribution. In the latter case, enhanced area-under-the-curve (AUC) would be achieved over the free drug even for the same elimination half life.

The invention also provides a method for improving the efficacy of a therapeutic agent comprising encapsulating the agent in a liposome, wherein, 1) the elimination half-life of the therapeutic agent when administered to an animal as part of the formulation is at least as long as the elimination half-life of the therapeutic agent when administered to the same animal in the absence of the liposome, and wherein 2) the elimination half-life of the therapeutic agent when administered as part of the formulation is less than about 14 hours in a rat.

The invention also provides a method for producing an anti-cancer (e.g. an antineoplastic) effect in an animal comprising administering to the animal an effective amount of a formulation of the invention wherein the therapeutic agent is an anti-cancer agent.

The invention also provides a method for producing an antibiotic effect in an animal comprising administering to the animal an effective amount of a formulation of the invention wherein the therapeutic agent is an antibiotic agent.

The invention also provides a formulation of the invention for use in medical therapy.

The invention also provides the use of a formulation of the invention wherein the therapeutic agent is an anti-cancer compound to prepare a medicament useful for producing an anti-cancer effect in a mammal.

The invention also provides the use of a formulation of the invention wherein the therapeutic agent is an antibiotic to prepare a medicament useful for producing an antibiotic effect in a mammal.

The invention also provides a pharmaceutical composition comprising a formulation of the invention, in combination with a pharmaceutically acceptable diluent or carrier.

The invention also provides processes and intermediated disclosed herein that are useful for preparing formulations of the invention.

### BRIEF DESCRIPTION OF THE FIGURES

FIG. 1 shows mouse survival data for liposomal formulations of cisplatin in Test C hereinbelow.

FIG. 2 shows the maximum tolerated dose of liposomal cisplatin in Test B hereinbelow.

FIG. 3 shows the efficacy of liposomal cisplatin and free cisplatin in Test C hereinbelow.

FIG. 4 shows plasma levels for liposomal formulations of cisplatin in Test A hereinbelow.

FIGS. 5-7 show plasma levels for liposomal formulations of amikacin in Test A hereinbelow.

FIG. 8 shows plasma levels for liposomal formulations of vancomycin in Test A hereinbelow.

FIG. 9 shows the effect of liposomal cisplatin (dosed at MTD) on human breast tumor MaTu growth in mice in Test D hereinbelow.

### DETAILED DESCRIPTION

The liposomes comprise a lipid layer comprising liposome forming lipids. Typically, the lipid includes at least one phosphatidyl choline which provides the primary packing/entrapment/structural element of the liposome. Typically, the phosphatidyl choline comprises mainly C₁₆ or longer fatty-acid chains. Chain length provides for both liposomal structure, integrity, and stability. Optionally, one of the fatty-acid chains have at least one double bond.

As used herein, the term “phosphatidyl choline” includes Soy PC, Egg PC dielaidoyl phosphatidyl choline (DEPC), dioleoyl phosphatidyl choline (DOPC), distearoyl phosphatidyl choline (DSPC), hydrogenated soybean phosphatidyl choline (HSPC), dipalmitoyl phosphatidyl choline (DPPC), 1-palmitoyl-2-oleo phosphatidyl choline (POPC), dibehenoyl phosphatidyl choline (DBPC), and dimyristoyl phosphatidyl choline (DMPC).

As used herein, the term “Soy-PC” refers to phosphatidyl choline compositions including a variety of mono-, di-, tri-unsaturated, and saturated fatty acids. Typically, Soy-PC includes palmitic acid present in an amount of about 12% to about 33% by weight; stearic acid present in an amount of about 3% to about 8% by weight; oleic acid present in an amount of about 4% to about 22% by weight; linoleic acid present in an amount of about 60% to about 66% by weight; and linolenic acid present in an amount of about 5% to about 8% by weight.

As used herein, the term “Egg-PC” refers to a phosphatidyl choline composition including, but not limited to, a variety of saturated and unsaturated fatty acids. Typically, Egg-PC comprises palmitic acid present in an amount of about 34% by weight; stearic acid present in an amount of about 10% by weight; oleic acid present in an amount of about 31% by weight; and linoleic acid present in an amount of about 18% by weight.

Cholesterol typically provides stability to the liposome. The ratio of phosphatidyl choline to cholesterol is typically from about 0.5:1 to about 4:1 by mole ratio. Preferably, the ratio of phosphatidyl choline to cholesterol is from about 1:1 to about 2:1 by mole ratio. More preferably, the ratio of phosphatidyl choline to cholesterol is about 2:1 by mole ratio.

As used herein the term “total lipid” includes phosphatidyl cholines and any anionic phospholipid present.

The liposome may also comprise physiologically acceptable salts to maintain isotonicity with animal serum. Any pharmaceutically acceptable salt that achieves isotonicity with animal serum is acceptable, such as NaCl.

In one embodiment, the liposome is not pegylated.

Preparation of Liposomes

The liposomes of the invention comprise a lipid layer of phospholipids and cholesterol. Typically, the ratio of phospholipid to cholesterol is sufficient to form a liposome that will not dissolve or disintegrate once administered to the animal. The phospholipids and cholesterol are dissolved in suitable solvent or solvent mixtures. After a suitable amount of time, the solvent is removed via vacuum drying and/or spray drying. The resulting solid material can be stored or used immediately.

Subsequently, the resulting solid material is hydrated in aqueous solution containing an appropriate concentration of the therapeutic agent at an appropriate temperature, resulting in multilameller vesicles (MLV). The solutions containing MLV can be size-reduced via homogenization to form Small Unilameller Vesicles (SUVs) with the drug passively entrapped within the formed SUVs. The resulting liposome solution can be purified of unencapsulated therapeutic agent, for example by chromatography or filtration, and then filtered for use.

Anionic Phospholipid

An anionic phospholipid may be used and typically provides a Coulombic character to the liposomes. This can help stabilize the system upon storage and can prevent fusion or aggregation or flocculation; it can also facilitate or enable freeze drying. Phospholipids in the phosphatidic acid, phosphatidylglycerol, and phosphatidylserine classes (PA, PG, and PS) are particularly useful in the formulations of the invention. The anionic phospholipids typically comprise mainly C₁₆ or larger fatty-acid chains.

In one embodiment the anionic phospholipid is selected from Egg-PG (Egg-Phosphatidyglycerol), Soy-PG (Soy-Phosphatidylglycerol), DSPG (Distearoyl Phosphatidyglycerol), DPPG (Dipalmitoyl Phosphatidyglycerol), DEPG (Dielaidoyl Phosphatidyglycerol), DOPG (Dioleoyl Phosphatidyglycerol), DSPA (Distearoyl Phosphatidic Acid), DPPA (Dipalmitoyl Phosphatidic Acid), DEPA (Dielaidoy Phosphatidic Acid), DOPA (Dioleoyl Phosphatidic Acid), DSPS (Distearoyl Phosphatidylserine), DPPS (Dipalmitoyl Phosphatidylserine), DEPS (Dielaidoy Phosphatidylserine), and DOPS (Dioleoyl Phosphatidylserine), and mixtures thereof.

In another embodiment the anionic phospholipid is DSPG.

Therapeutic Agents

Many highly active and useful pharmaceutical agents suffer from sub-optimal pharmacokinetics and/or biodistribution. Consequently, the therapeutic use of these pharmaceutical agents can be limited. Liposome dispersions of the invention can be used to improve the efficacy or toxicity profiles or both, or to improve the dosing schedule of the drug by modification of the pharmacokinetic/biodistribution. As used herein, the term therapeutic agent includes diagnostic agents.

The term “lipophobic therapeutic agent” includes compounds that are water soluble enough to achieve a useful level of loading by passive encapsulation and that are significantly impermeable once loaded. The term excludes agents that are both amphiphilic and that can be effectively gradient loaded into liposomes. Accordingly, the formulations of the invention are typically prepared by passive loading of liposomes.

The term therapeutic agent includes but is not limited to, an analgesic, an anesthetic, an antiacne agent, an antibiotic, an antibacterial, an anticancer, an anticholinergic, an anticoagulant, an antidyskinetic, an antiemetic, an antifibrotic, an antifungal, an antiglaucoma agent, an anti-inflammatory, an antineoplastic, an antiosteoporotic, an antipagetic, an anti-Parkinson's agent, an antisporatic, an antipyretic, an antiseptic, an antithrombotic, an antiviral, a calcium regulator, a keratolytic, or a sclerosing agent.

In one embodiment the therapeutic agent is an anti-cancer agent, an antibiotic (e.g. an aminoglycoside or a glycopeptide), a nucleoside, a nucleotide, DNA, RNA, a protein or a peptide. In another embodiment the therapeutic agent is an antineoplastic agent. In yet another embodiment the therapeutic agent is cisplatin, a cisplatin derivative, amikacin, or vancomycin.

Cisplatin Derivatives

In one embodiment the therapeutic agent can be native cisplatin and in another embodiment, the therapeutic agent can be a cisplatin derivative, preferably a hydrophilic cisplatin derivative.

Native cisplatin, also referred to herein as cisplatin, is a heavy metal complex containing a central atom of platinum surrounded by two chloride atoms and two ammonia molecules in the cis position. It is a yellow powder with a molecular weight of 300.1. It is soluble at room temperature in water or saline at 1 mg/ml and has a melting point of 207° C.

The chlorine atoms in cisplatin are subject to chemical displacement reactions by nucleophiles, such as water or sulfhydryl groups. In aqueous media, water molecules are potential ligands, which may replace the chlorine atoms to form monohydroxymonochloro cis-diamine platinum (II).

The drug is available as a sterile aqueous solution containing 1 mg cisplatin and 9 mg NaCl per ml water and in this form is typically administered intravenously for tumor therapy at a dose of between about 20-120 mg/m². The drug may be administered alone or in combination with other chemotherapeutic agents, as a bolus injection or as a slow infusion over a period of several hours.

As a single agent, cisplatin can be administered, for example, at a dose of 100 mg/m² intravenously once every 4 weeks or at a dose of 20 mg/m² cisplatin given as a rapid intravenous infusion daily for 5 days and repeated at 4-week intervals.

While active as a single agent, cisplatin is often administered in combination with other agents, including vinblastine, bleomycin, actinomycin, adriamycin, prednisone, vincristine, and others. For example, therapy of ovarian cancer may include 60 mg/m² cisplatin and 60 mg/m² adriamycin administered as a 24-hour infusion.

In another embodiment of the invention, the cisplatin compound entrapped within the liposomes is a cisplatin derivative. Numerous cisplatin derivatives have been synthesized. Such analogues include carboplatin, ormaplatin, oxaliplatin, DWA21 14R ((−)-(R)-2-aminomethylpyrrolidine (1,1-cyclobutane dicarboxylato)platinum), zeniplatin, enloplatin, lobaplatin, CI-973 (SP-4-3(R)-1,1-cyclobutane-dicarboxylato(2−)-(2-methyl-1,4-butanediamine-N,N′)platinum), 254-S nedaplatin and JM-216 (bis-acetato-ammine-dichloro-cyclohexylamine-platinum(IV). Some cisplatin analogues, such as spiroplatin, have been found to be more toxic than native cisplatin. While more toxic analogues are not desirable for intravenous administration in free form, such analogues may have use in liposome-entrapped form, which reduces drug toxicity.

For purposes of the present invention, analogues having some water solubility, such as carboplatin, iproplatin and others, may be preferred so that the drug is entrapped primarily in the inner aqueous compartment of the liposome.

In one embodiment the cisplatin analogue is carboplatin, (1,1-cyclobutane-dicarboxylate-diammineplatinum), which contains organic ligands in a 4-coordinate planar complex of platinum.

Relative Amounts

In one embodiment the lipid-based dispersion comprises from 0.05 to 60% anionic phospholipid by molar ratio relative to phosphatidyl choline.

In one embodiment the weight ratio of total lipid (phosphatidyl choline+anionic phospholipid) to therapeutic agent is greater than 1:1.

In another embodiment the weight ratio of total lipid (phosphatidyl choline+anionic phospholipid) to therapeutic agent is greater than 5:1.

In another embodiment the weight ratio of total lipid (phosphatidyl choline+anionic phospholipid) to therapeutic agent is greater than 10:1.

In another embodiment the weight ratio of total lipid (phosphatidyl choline+anionic phospholipid) to therapeutic agent is greater than 20:1.

In one embodiment, the invention provides a formulation comprising a lipophobic therapeutic agent in a liposome that comprises HSPC:Cholesterol:DSPG in a ratio of about 4:1:0.1.

In another one embodiment, the invention provides a formulation comprising a lipophobic therapeutic agent in a liposome that comprises DEPC:Cholesterol in a ratio of about 2:1.

In another one embodiment, the invention provides a formulation comprising a lipophobic therapeutic agent in a liposome that comprises DEPC:Cholesterol:DSPG in a ratio of about 2:1:0.1.

In another one embodiment, the invention provides a formulation comprising a lipophobic therapeutic agent in a liposome that comprises DOPC:Cholesterol in a ratio of about 2:1.

In another one embodiment, the invention provides a formulation comprising a lipophobic therapeutic agent in a liposome that comprises DMPC:Cholesterol:DSPG in a ratio of about 2:1:0.1.

Formulations

The formulations of the invention can be administered to a mammalian host, such as a human patient in a variety of forms adapted to the chosen route of administration. For example, they can be formulated to be administered parenterally. Moreover, the lipid-based dispersions can be formulated for subcutaneous, intramuscular, intravenous, or intraperitoneal administration by infusion or injection. These preparations may also contain a preservative to prevent the growth of microorganisms, buffers, or anti-oxidants in suitable amounts.

Useful dosages of the formulations of the invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of a therapeutic agent in a unit dosage form of the invention will typically be from about 0.5-50% by weight of the composition, preferably from about 2-20% by weight of the composition.

The amount of therapeutic agent required for use in treatment will vary not only with particular agent but also with the route of administration, the nature of the condition being treated and the age and condition of the patient; the amount required will be ultimately at the discretion of the attendant physician or clinician.

The desired amount of a formulation may conveniently be presented in a single dose or as divided doses administered at appropriate intervals, for example, as two, three, four or more sub-doses per day. The sub-dose itself may be further divided, e.g., into a number of discrete loosely spaced administrations.

In one embodiment, the invention provides a formulation comprising a lipophobic therapeutic agent encapsulated in a liposome, wherein, 1) the elimination half-life of the therapeutic agent when administered to an animal as part of the formulation is at least about 1.5-times as long as the elimination half-life of the therapeutic agent when administered to the same animal in the absence of the liposome, and wherein 2) the elimination half-life of the therapeutic agent when administered as part of the formulation is less than about 14 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 2-times as long as the elimination half-life of the therapeutic agent when administered to the same animal in the absence of the liposome, and wherein 2) the elimination half-life of the therapeutic agent when administered as part of the formulation is less than about 14 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 3-times as long as the elimination half-life of the therapeutic agent when administered to the same animal in the absence of the liposome, and wherein 2) the elimination half-life of the therapeutic agent when administered as part of the formulation is less than about 14 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 1.5-times as long as the elimination half-life of the therapeutic agent when administered to the same animal in the absence of the liposome, and wherein 2) the elimination half-life of the therapeutic agent when administered as part of the formulation is less than about 12 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 2-times as long as the elimination half-life of the therapeutic agent when administered to the same animal in the absence of the liposome, and wherein 2) the elimination half-life of the therapeutic agent when administered as part of the formulation is less than about 12 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 3-times as long as the elimination half-life of the therapeutic agent when administered to the same animal in the absence of the liposome, and wherein 2) the elimination half-life of the therapeutic agent when administered as part of the formulation is less than about 12 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 1.5-times as long as the elimination half-life of the therapeutic agent when administered to the same animal in the absence of the liposome, and wherein 2) the elimination half-life of the therapeutic agent when administered as part of the formulation is less than about 10 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 2-times as long as the elimination half-life of the therapeutic agent when administered to the same animal in the absence of the liposome, and wherein 2) the elimination half-life of the therapeutic agent when administered as part of the formulation is less than about 10 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 3-times as long as the elimination half-life of the therapeutic agent when administered to the same animal in the absence of the liposome, and wherein 2) the elimination half-life of the therapeutic agent when administered as part of the formulation is less than about 10 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 1.5-times as long as the elimination half-life of the therapeutic agent when administered to the same animal in the absence of the liposome, and wherein 2) the elimination half-life of the therapeutic agent when administered as part of the formulation is less than about 8 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 2-times as long as the elimination half-life of the therapeutic agent when administered to the same animal in the absence of the liposome, and wherein 2) the elimination half-life of the therapeutic agent when administered as part of the formulation is less than about 8 hours in a rat.

In one embodiment, the invention provides a formulation comprising a lipophobic therapeutic agent encapsulated in a liposome, wherein, 1) the elimination half-life of the therapeutic agent when administered to an animal as part of the formulation is at least about 3-times as long as the elimination half-life of the therapeutic agent when administered to the same animal in the absence of the liposome, and wherein 2) the elimination half-life of the therapeutic agent when administered as part of the formulation is less than about 8 hours in a rat.

Pharmacokinetic data (plasma concentration vs. time post injection) for a therapeutic agent in a formulation of the invention and for the free therapeutic agent can be determined in an array of known animal models. For example, it can be determined in rats using Test A.

Test Method A—Pharmacokinetics (PK)

Pharmacokinetic data (plasma concentration vs. time post injection) were obtained for one dose per liposome formulation and the corresponding free drug. Sprague Dawley or Wistar rats, female, were used, weighing about 150 g. Typically there were 6 rats per dose group. Plasma pulls of 200 microliters (sampling from the orbital sinus) were collected in EDTA tubes, with samples frozen prior to chemical analysis of the drug. Elimination half life is determined by fitting the data against a single or double exponential decay equation.

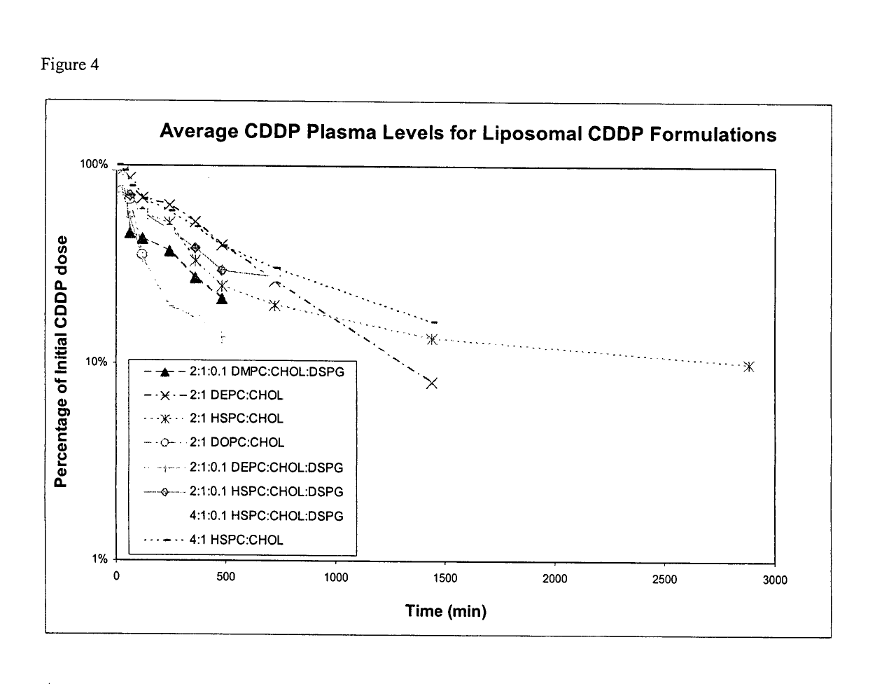

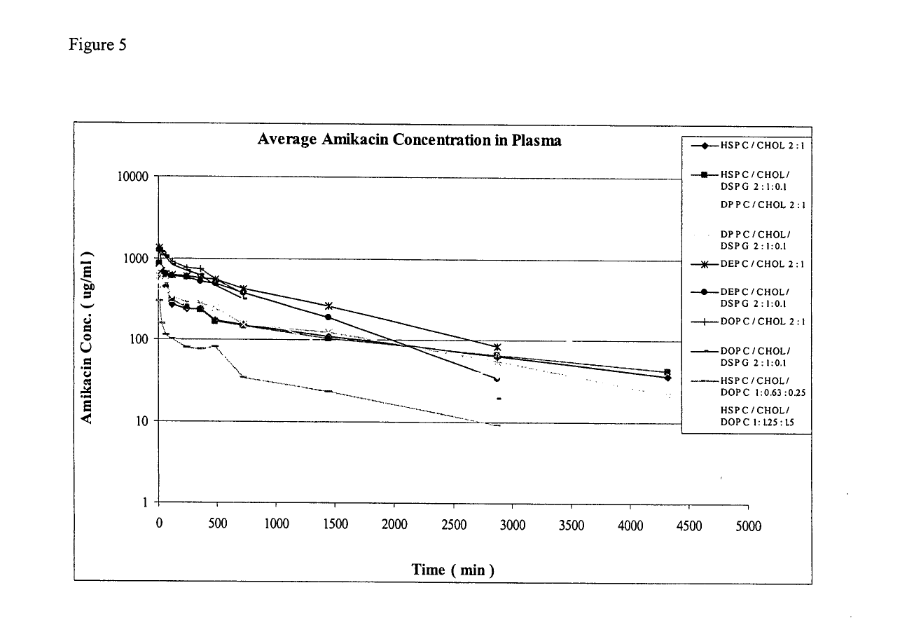

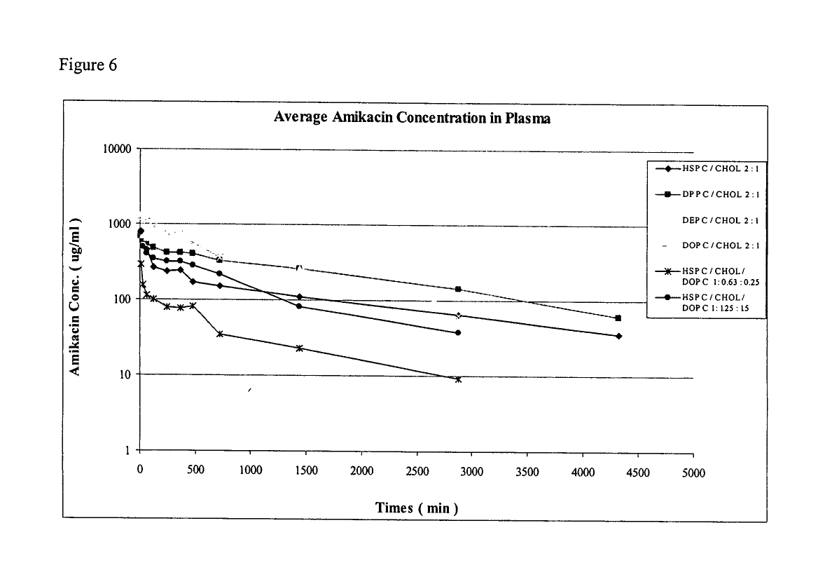

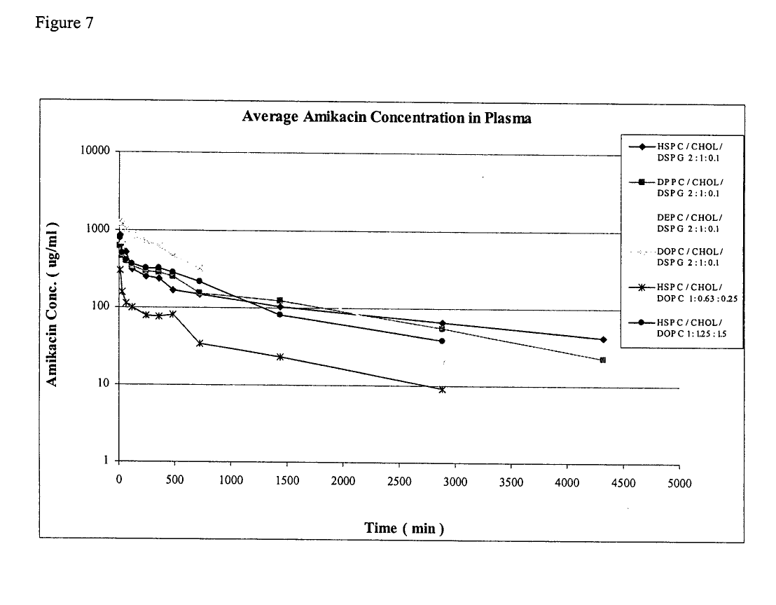

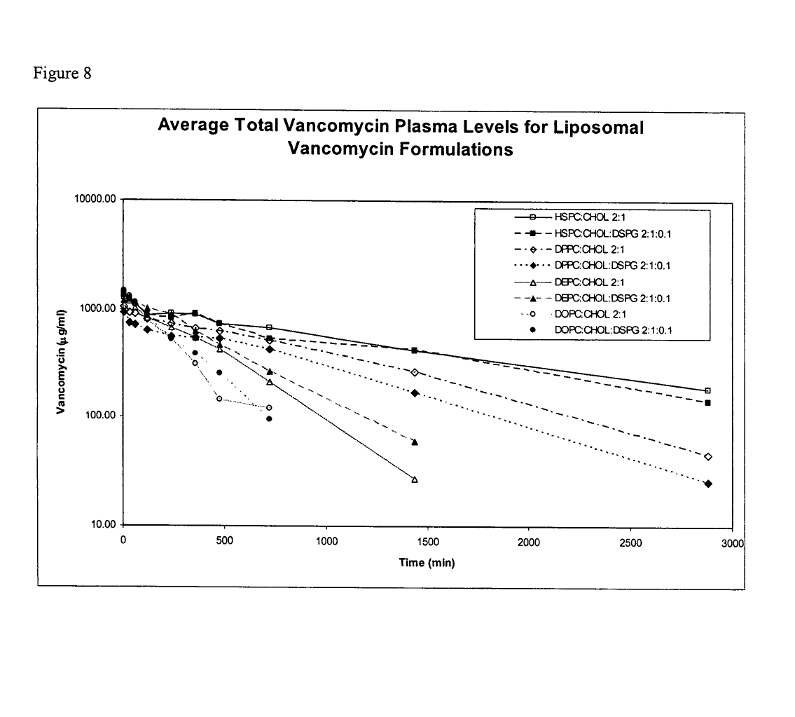

Representative plasma concentrations for formulations comprising, cisplatin are shown in FIG. 4; amikacin are shown in FIGS. 5-7; and vancomycin are shown in FIG. 8.

The elimination half-lives for various cicplatin (CDDP) formulations are shown in the following table.

| CDDP | Elimination Half |
|---|---|
| Formulation | Life (Hours) |
| HSPC:CHOL (2:1) | 15-20 |
| HSPC/CHOL/DSPG (2:1:0.1) | 15-20 |
| HSPC/CHOL (4:1) | 6.6 |
| DOPC/CHOL (2:1) | 1 |
| DEPC:CHOL (2:1) | 3 |
| HSPC/CHOL/DSPG (4:1:0.1) | 3.9 |
| DEPC/CHOL/DSPG (2:1:0.1) | 3.6 |
| DMPC:CHOL:DSPG (2:1:0.1) | 1-4 |

The elimination half-lives for various amikacin formulations are shown in the following table.

| Amikacin | Elimination Half |
|---|---|
| Formulation | Life (Hours) |
| HSPC:CHOL 2:1 | 14.7 |
| HSPC:CHOL:DSPG 2:1:0.1 | 10.8 |
| DPPC:CHOL 2:1 | 10.8 |
| DPPC:CHOL:DSPG 2:1:0.1 | 11.0 |
| DEPC:CHOL 2:1 | 23.4 |
| DEPC:CHOL:DSPG 2:1:0.1 | 17.6 |
| DOPC:CHOL 2:1 | 7.5 |
| DOPC:CHOL:DSPG 2:1:0.1 | 7.2 |
| HSPC:CHOL:DOPC 1:0.63:0.25 | 9.1 |
| HSPC:CHOL:DOPC 1:1.25:1.5 | 13.3 |
| HSPC:CHOL:DMPC 1:0.63:0.25 | 16.6 |
| HSPC:CHOL:DMPC 1:1.25:1.5 | 12.9 |

The elimination half-lives for various vancomycin formulations are shown in the following table:

| Vancomycin | Elimination Half |
|---|---|
| Formulation | Life (Hours) |
| HSPC:CHOL 2:1 | 20.3 |
| HSPC:CHOL:DSPG 2:1:0.1 | 17.5 |
| DPPC:CHOL 2:1 | 17.0 |
| DPPC:CHOL:DSPG 2:1:0.1 | 16.9 |
| DEPC:CHOL 2:1 | 6.4 |
| DEPC:CHOL:DSPG 2:1:0.1 | 6.3 |
| DOPC:CHOL 2:1 | 2.2 |
| DOPC:CHOL:DSPG 2:1:0.1 | 2.6 |

The maximum tolerated dose for a therapeutic agent in a formulation of the invention and for the free therapeutic agent can be determined in an array of known animal models. For example, it can be determined using Test B.

Test Method B—Maximum Tolerated Dose (MTD)

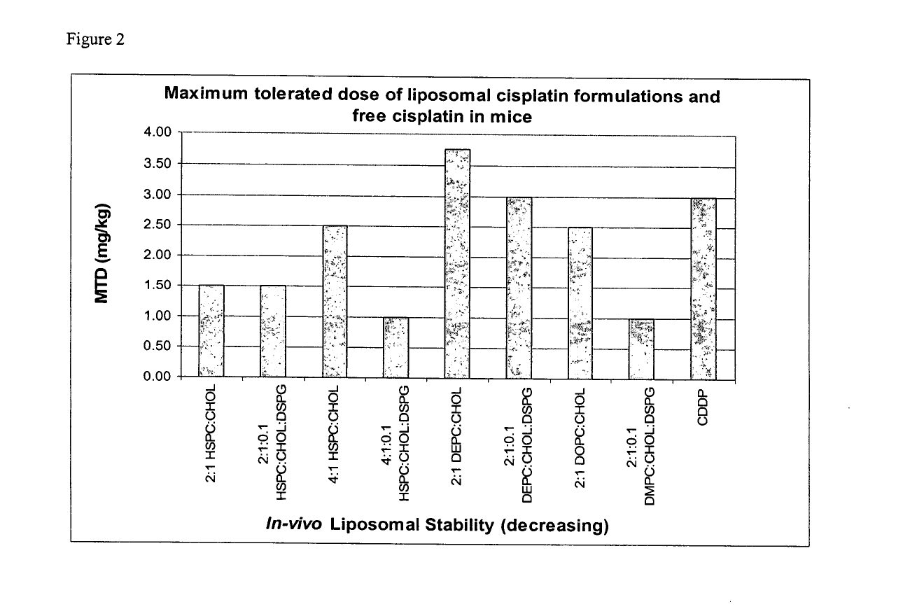

Nude mice (NCr.nu/nu—mice) were administered each liposomal formulation, and free drug, by I.V. administration and the maximum tolerated dose (MTD) for each formulation was then determined. Typically a range of doses were given until an MTD was found, with 2 mice per dose group. Estimate of MTD was determined by evaluation of body weight, lethality, behavior changes, and/or signs at autopsy. Typical duration of the experiment is observation of the mice for four weeks, with body weight measurements twice per week. Data for formulations comprising cisplatin are shown in FIG. 2.

The anti-leukemia activity for a therapeutic agent in a formulation of the invention and for the free therapeutic agent can be determined in an array of known animal models. For example, it can be determined in rats using Test C.

Test Method C—P-388Leukemia Efficacy

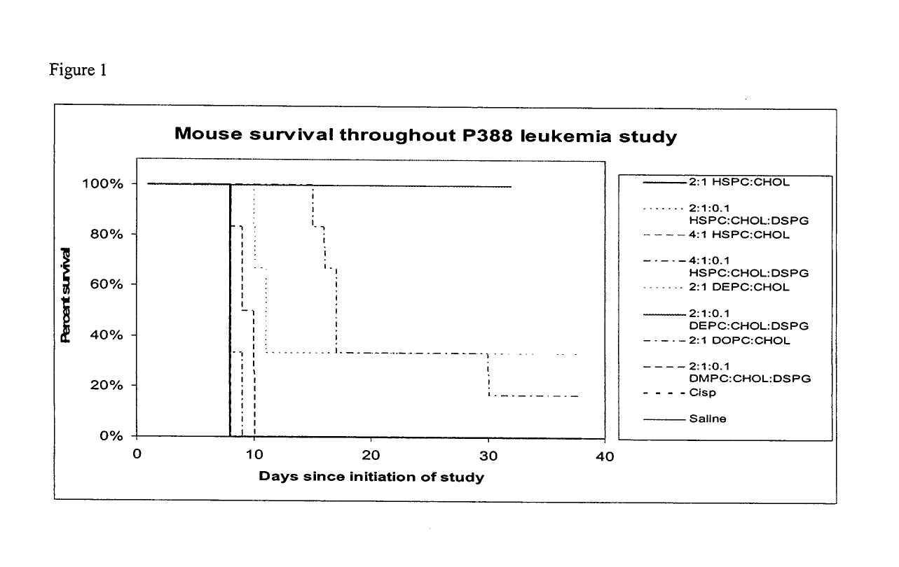

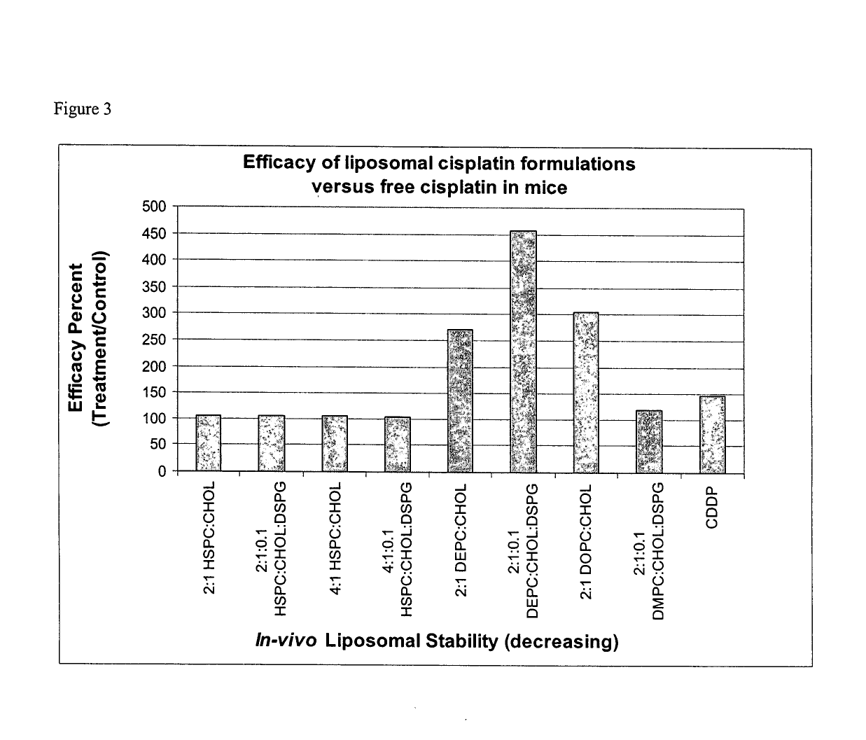

B6D2F-1 mice (6 per group) were injected with cells from a P388 leukemia cell line (B-lymphatic leukemia P388, 106 cells/mouse i.v. on day zero). Mice were treated typically on day one or on days one, two and three at the MTD previously determined for each formulation and for free drug. Efficacy was calculated as the percentage increase in median survival time of the mice treated with a specific test article versus those mice treated with the control (saline). Duration of the experiment is typically 3-4 weeks (or if long term survivors occur, 45 days). Representative data for formulations comprising cisplatin are shown in FIG. 1 and FIG. 3.

The anti-cancer activity for a therapeutic agent in a formulation of the invention and for the free therapeutic agent can be determined in an array of known animal models. For example, it can be determined in rats using Test D.

Test Method D—Breast Cancer Xenograft Models

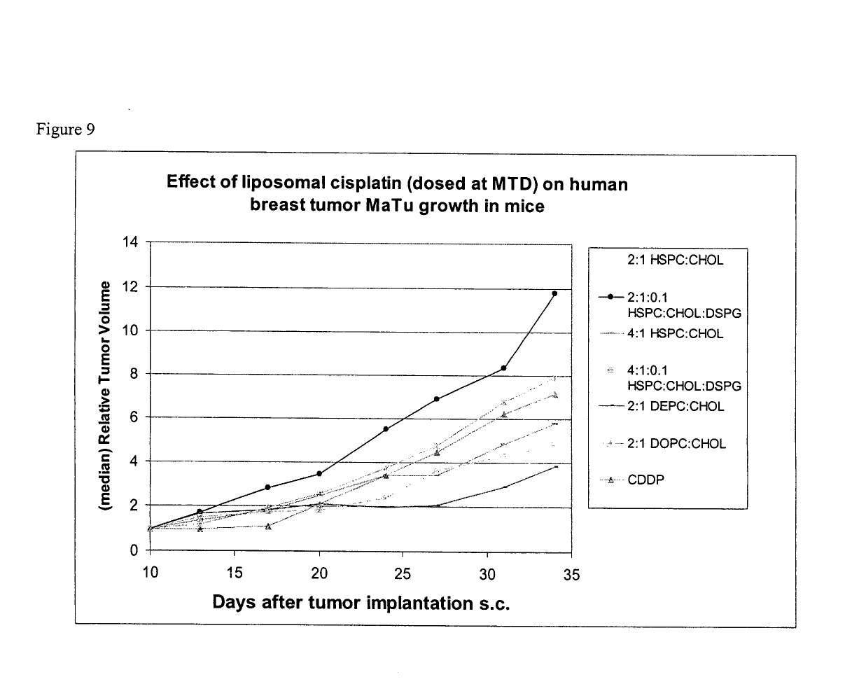

Nude mice were subcutaneously implanted with MaTu or MT-3 human breast carcinoma cells and were subsequently treated with liposomal formulations in addition to free drug and a saline control. Treatment began on the tenth day after tumor implantation and consisted of dosing animals once or once a day for three consecutive days at the MTD of each respective agent. Tumor volumes were measured at several time points throughout the study with the study terminating about thirty-four days after tumor implantation. The median relative tumor volume (each individual tumor size measurement as related to the size of the tumor that was measured on day ten of the study) is plotted for each of the test articles. Representative data for formulations comprising cisplatin are shown in FIG. 9. Of the six liposomal formulations tested in the breast cancer model, four showed a greater reduction in tumor volume than the cisplatin control.

The invention is further defined by reference to the following examples describing the preparation of formulations of the invention. It will be apparent to those skilled in the art, that many modifications, both to materials and methods, may be practiced without departing from the purpose and interest of this invention.

### EXAMPLES

General Procedure of Liposome Preparation

Lipid films or lipid spray dried powder containing various phospholipids including hydrogenated soy phosphatidyl choline (HSPC), dioleoyl phosphatidyl choline (DOPC), dielaidoyl phosphatidyl choline (DEPC), cholesterol (Chol) and distearoylphosphatidylglycerol (DSPG) at the following mole ratios were prepared.

HSPC:Chol:DSPG at a) 2:1:0 b) 2:1:0.1 c) 4:1:0 d) 4:1:0.1

DOPC:Chol:DSPG at a) 2:1:0 b) 2:1:0.1

DEPC:Chol:DSPG at a) 2:1:0 b) 2:1:0.1

DMPC:Chol:DSPG at a) 2:1:0.1

Lipid Film Preparation.

Stock solution of each lipid component was made in a chloroform: methanol 1:1 (v/v) organic solvent system. The final concentration of each lipid component was: HSPC, DOPC, DEPC and Chol (200 mg/ml); and DSPG (50 mg/ml). Lipid solutions were pipetted according to the designed mole ratio and were mixed in a conical tube. The final lipid concentration was around 200 mg/ml. The solvent was then removed by running nitrogen through the solution while the solution was heated in heat block with temperature set at 65 C. The formed lipid film was then left in desiccator under vacuum to remove residual organic solvent till being used.

Spray Dried Lipid Powder Preparation

All the lipid component were weighed out and were mixed in a round bottom flask, a chloroform:methanol 1:1 (v/v) solvent was added to the lipid powder with a final lipid concentration around 200 mg/ml. The lipid solution was then spray dried to form lipid powder using a YAMATO GB-21 spray drier at a designed parameter setting. The residual solvent in the lipid powder was removed by drying under vacuum for three to five days.

Cis-Platinum (CDDP) Stock Solution Preparation

Cis-platinum powder was weighted out, a 200 mM sodium chloride solution pH=6.4 was added to the drug powder to make a final CDDP stock solution at 10 mg/ml. The CDDP drug solution was exposed to probe sonication at 70° C. for around 2 to 3 minutes to ensure that all entire drug is dissolved. The stock solution was then kept in a 70° C. water bath to maintain a clear, precipitation-free solution.

Preparation of Liposomes by Probe Sonication from Either Lipid Film or Spray Dried Lipid Powder

Lipid film or lipid powder was weighed out and hydrated with CDDP stock solution in a 70° C. water bath at lipid concentration approximately 150 mg/ml. The hydrated solution was subjected to probe sonication until the solution became translucent. A typical temperature of sonication was 70° C. and a typical sonication time was 15 to 20 minutes. After completion of sonication, the liposomes were subjected to one of the following cleaning procedures: a) the liposomes were cooled down to ambient temperature for around 4 hours, and the yellow precipitation was removed by centrifugation, and the precipitation-free clear solution was applied to a sephadex G-50 column for buffer exchange with 9% sucrose; or b) upon completion of sonication, the liposomal solution was immediately diluted one to ten with 200 mM sodium chloride solution; that diluted solution was subjected to ultra filtration for cleaning/buffer exchange with 9% sucrose; and the sterilization filtration of the liposome solution was made at ambient temperature through a cellulose acetate 0.22 micron filter.

Preparation of Liposomes by Homogenization from Spray Dried Lipid Powder

Lipid powder was weighed out and were hydrated with CDDP stock solution in a 70° C. water bath at lipid concentration approximately 100 mg/ml. The hydrated solution was subjected to homogenization using a Niro homogenizer at 10,000 PSI at 70 C until the solution became translucent. A typical homogenization process took about 20 passes. After completion of homogenization, the liposomal solution was immediately diluted one to ten with 200 mM sodium chloride solution. That diluted solution was then subjected to ultra filtration for cleaning/buffer exchange with 9% sucrose. The sterilization filtration of the liposome solution was made at ambient temperature through a cellulose acetate 0.22 micron filter.

### Example 1

### Liposomes Containing Cisplatin were Prepared as Described Above

Characterization data for representative liposomes is shown in the following table.

| | Lipid | Mole | | Size | | |
|---|---|---|---|---|---|---|
| Number | Formulation | Ratio | A600 | (nm) | Volume % | pH |
| 1 | HSPC/Chol | 2:1 | 0.699 | 51.7 | 100 | |
| 2 | HSPC/Chol/DSPG | 2:1:0.1 | 0.368 | 45.4 | 100 | |
| 3 | HSPC/Chol | 4:1 | 0.894 | 52.8 | 100 | 5.59 |
| 4 | DOPC/Chol | 2:1 | 0.224 | 42.2 | 100 | 4.87 |
| 5 | DEPC/Chol | 2:1 | 0.211 | 31.1 | 100 | 4.83 |
| 6 | HSPC/Chol/DSPG | 4:1:0.1 | 0.613 | 42.4 | 100 | 5.46 |
| 7 | DEPC/Chol/DSPG | 2:1:0.1 | 0.240 | 35.0 | 100 | 5.58 |
| 8 | DMPC/Chol/DSPG | 2:1:0.1 | 0.473 | 37.0 | 100 | 5.62 |
| 9 | HSPC/Chol | 2:1 | 1.310 | 43.9 | 100 | 6.55 |
| 10 | HSPC/Chol/DSPG | 2:1:0.1 | 0.815 | 43.7 | 100 | 6.39 |
| 11 | HSPC/Chol | 4:1 | 1.922 | 63.4 | 100 | 7.04 |
| 12 | DOPC/Chol | 2:1 | 0.493 | 41.1 | 100 | 6.72 |
| 13 | DEPC/Chol | 2:1 | 1.179 | 30.5 | 100 | 6.37 |
| 14 | HSPC/Chol/DSPG | 4:1:0.1 | 0.753 | 61.4 | 100 | 6.66 |
| 15 | DEPC/Chol/DSPG | 2:1:0.1 | 0.277 | 29.2 | 100 | 6.00 |
| 16 | DMPC/Chol/DSPG | 2:1:0.1 | 0.502 | 40.0 | 100 | 5.68 |
| 17 | DEPC/Chol | 2:1 | 1.143 | 39.9 | 100 | 7.05 |
| 18 | HSPC/Chol/DSPG | 0 | 0.868 | 33.9 | 100 | 5.18 |
| 19 | DEPC/Chol/DSPG | 2:1:0.1 | 0.960 | 41.8 | 100 | 6.10 |
| 20 | HSPC/Chol/DSPG | 4:1:0.1 | 0.648 | 27.4 | 100 | 6.28 |
| 21 | DEPC/Chol/DSPG | 2:1:0.1 | 0.270 | 31.1 | 100 | 5.20 |
| 22 | HSPC/Chol | 4:1 | 1.858 | 78.6 | 100 | 5.75 |
| 23 | DOPC/Chol | 2:1 | 0.304 | 38.2 | 100 | 5.24 |
| 24 | DEPC/Chol | 2:1 | 0.905 | 35.6 | 100 | 6.31 |
| 25 | DOPC/Chol/DSPG | 2:1:0.1 | 0.182 | 39.5 | 81 | 5.50 |
| 26 | DOPC/Chol | 2:1 | 0.189 | 50.4 | 100 | 5.49 |

### Example 2

### Liposomes Containing Amikacin were Prepared as Follows

Preparation of Amikacin (AMK) Stock Solution

Amikacin free base powder was weighted out and was mixed with water for injection (WFI). The pH of the Amikacin slurry was titrated to around pH 6.5. The final volume of the stock solution was brought up by addition of WFI. The final concentration of the Amikacin stock solution was around 250 mg/ml with final pH of around 6.5.

Preparation of Liposome by Probe Sonication from Either Lipid Film or Spray Dried Lipid Powder

A proper amount of lipid was weighted out. The lipid was hydrated with Amikacin stock solution at 300 mg/ml lipid concentration. The mixture was then incubated at 65° C. for around 10-20 minutes and sonicated at around 60° C. for 20 minutes or until the solution became transparent. Upon completion of sonication, the liposome solution was diluted 1:1 with 10 mM sodium Succinate in 9% Sucrose pH=6.5. The post diluted liposome solution was then passed through sephadex column to remove free drug by buffer exchanging with 10 mM sodium Succinate in 9% Sucrose pH=6.5. The liposomes were filtered at ambient temperature through a cellulose acetate 0.22 micron filter. Characterization data for representative liposomes is shown in the following table.

| Lipid | Mole | | Size | | |
|---|---|---|---|---|---|
| Formulation | Ratio | A600 | (nm) | Volume % | pH |
| HSPC/Chol | 2:1 | 1.553 | 63.3 | 100 | 6.68 |
| HSPC/Chol/DSPG | 2:1:0.1 | 1.347 | 59.2 | 100 | 6.71 |
| DPPC/Chol/DSPG | 2:1:0.1 | 1.111 | 52.9 | 100 | 6.84 |
| DEPC/Chol | 2:1 | 1.161 | 54.1 | 100 | 6.41 |
| DEPC/Chol/DSPG | 2:1:0.1 | 1.075 | 47.1 | 100 | 6.54 |
| DOPC/Chol | 2:1 | 1.085 | 78.7 | 100 | 6.56 |
| DOPC/Chol/DSPG | 2:1:0.1 | 0.693 | 67.1 | 100 | 6.20 |
| DPPC/Chol | 2:1 | 1.323 | 57.3 | 100 | 6.51 |
| HSPC/Chol/DOPC | 1:0.63:0.25 | 2.074 | 71.5 | 100 | 6.38 |
| HSPC/Chol/DOPC | 1:1.25:1.5 | 1.138 | 82.5 | 100 | 6.41 |
| HSPC/Chol/DMPC | 1:0.75:0.5 | 2.337 | 64.2 | 96 | 6.32 |
| HSPC/Chol/DMPC | 1:1.25:1.5 | 2.257 | 63.3 | 100 | 6.46 |

### Example 3

### Liposomes Containing Vancomycin were Prepared as Follows

Preparation of Vancomycin (VANCO) Stock Solution

Vancomycin hydrochloride powder was weighted out and was mixed with proper amount of 0.15M hydrochloride (HCl) solution. The slurry was heated at 65° C. water bath to ensure the entire drug dissolved. Q.S the final volume of the stock solution to make the concentration about 300 mg/ml and the pH of the stock solution around 2.4.

Preparation of Liposome by Probe Sonication from Either Lipid Film or Spray Dried Lipid Powder

A proper amount of lipid was weighted out. The lipid was hydrated with Vancomycin stock solution at 300 mg/ml lipid concentration. The mixture was sonicated at around 60° C. for 20 minutes or until the solution became transparent. Upon completion of sonication, the liposome solution was diluted 1:1 with acidic 9% Sucrose. The post diluted liposome solution was then passed through sephadex column to remove free drug by buffer exchanging with 10 mM Ammonium Chloride in 9% Sucrose pH=6.5. The liposomes were filtered at ambient temperature through a cellulose acetate 0.22 micron filter. Characterization data for representative liposomes is shown in the following table.

| Lipid | Mole | | Size | | |
|---|---|---|---|---|---|
| Formulation | Ratio | A600 | (nm) | Volume % | pH |
| DPPC/Chol | 2:1 | 1.566 | 42.9 | 100 | 5.82 |
| DPPC/Chol/DSPG | 2:1:0.1 | 0.505 | 31.1 | 100 | 5.90 |
| HSPC/Chol | 2:1 | 2.569 | 75.3 | 100 | 6.64 |
| HSPC/Chol/DSPG | 2:1:0.1 | 2.515 | 64.3 | 100 | 6.59 |
| DEPC/Chol | 2:1 | 1.343 | 28.1 | 100 | 5.98 |
| DEPC/Chol/DSPG | 2:1:0.1 | 0.862 | 34.3 | 100 | 6.31 |
| DOPC/Chol | 2:1 | 0.615 | 31.1 | 100 | 5.81 |
| DOPC/Chol/DSPG | 2:1:0.1 | 0.886 | 35.0 | 100 | 5.79 |

### Example 4

The following illustrate representative pharmaceutical dosage forms, containing a lipid-based dispersion of the invention, for therapeutic or prophylactic use in animals (e.g. humans).

mg/ml

(i) Injection 1 (1 mg/ml)

| | |
|---|---|
| ‘Therapeutic Agent’ | 1.0 |
| Phosphatidyl choline | 40 |
| Cholesterol | 10 |
| Sucrose | 90 |
| 0.1 N Sodium hydroxide solution | |
| (pH adjustment to 7.0-7.5) | q.s. |
| Water for injection | q.s. ad 1 mL |

(ii) Injection 2 (10 mg/ml)

| | |
|---|---|
| ‘Therapeutic Agent’ | 10 |
| Phosphatidyl choline | 60 |
| Cholesterol | 15 |
| Anionic Phospholipid | 3 |
| 0.1 N Sodium hydroxide solution | |
| (pH adjustment to 7.0-7.5) | q.s. |
| sucrose | 90 |
| Water for injection | q.s. ad 1 mL |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A formulation comprising a lipophobic therapeutic agent encapsulated in a liposome, wherein, 1) the elimination half-life of the therapeutic agent when administered to an animal as part of the formulation is at least as long as the elimination half-life of the therapeutic agent when administered to the same animal in the absence of the liposome, and wherein 2) the elimination half-life of the therapeutic agent when administered as part of the formulation is less than about 14 hours in a rat.

2. The formulation of claim 1 wherein the liposome comprises a) one or more phosphatidyl choline; b) cholesterol; and optionally c) one or more anionic phospholipids.

3. The formulation of claim 1 wherein the therapeutic agent is an analgesic, anesthetic, antiacne agent, antibiotic, antibacterial, anticancer, anticholinergic, anticoagulant, antidyskinetic, antifibrotic, antifungal, antiglaucoma agents, anti-inflammatory, antineoplastic, antiosteoporotic, antipagetic, anti-Parkinson's agent, antisporatic, antipyretic, antiseptic, antithrombotic, calcium regulator, keratolytic, or a sclerosing agent.

4. The formulation of claim 1 wherein the therapeutic agent is an anti-cancer agent, an antibiotic, a nucleoside, a nucleotide, DNA, RNA, a protein, or a peptide.

5. The formulation of claim 1 wherein the therapeutic agent is cisplatin, a cisplatin derivative, amikacin, or vancomycin.

6. The formulation of claim 2 wherein the mole ratio of phosphatidyl choline to cholesterol is from about 0.5 to 1 to about 4:1.

7. The formulation of claim 2 wherein the mole ratio of phosphatidyl choline to cholesterol is from about 1 to 1 to about 2:1

8. The formulation of claim 2 wherein the mole ratio of phosphatidyl choline to cholesterol is about 2:1.

9. The formulation of claim 2 wherein the phosphatidyl choline is selected from DEPC, DOPC, DSPC, HSPC, DMPC, and DPPC, and mixtures thereof.

10. The formulation of claim 2 wherein the phosphatidyl choline is selected from DOPC, DSPC, HSPC, DMPC, and DPPC, and mixtures thereof.

11. The formulation of claim 2 wherein the phosphatidyl choline is selected from DOPC, DSPC, HSPC, and DPPC, and mixtures thereof.

12. The formulation of claim 2 wherein the phosphatidyl choline is DEPC or DOPC.

13. The formulation of claim 1 wherein the liposome is an SUV or an MLV.

14. The formulation of claim 1 wherein the mean particle size measured by dynamic light scattering is less than about 100 nm.

15. The formulation of claim 1 wherein the animal is a mammal.

16. The formulation of claim 1 wherein the animal is a mouse, a dog or a primate.

17. The formulation of claim 1 wherein the animal is a human.

18. The formulation of claim 1 wherein the weight ratio of total lipid to therapeutic agent is greater than 5:1.

19. The formulation of claim 1 wherein the weight ratio of total lipid to therapeutic agent is greater than 10:1.

20. The formulation of claim 1 wherein the weight ratio of total lipid to therapeutic agent is greater than 20:1.

21. The formulation of claim 1 wherein the elimination half-life of the therapeutic agent when administered to an animal as part of the formulation is at least about 1.5-times as long as the elimination half-life of the therapeutic agent when administered to the same animal in the absence of the liposome.

22. The formulation of claim 1 wherein the elimination half-life of the therapeutic agent when administered to an animal as part of the formulation is at least about 2-times as long as the elimination half-life of the therapeutic agent when administered to the same animal in the absence of the liposome.

23. The formulation of claim 1 wherein the elimination half-life of the therapeutic agent when administered to an animal as part of the formulation is at least about 3-times as long as the elimination half-life of the therapeutic agent when administered to the same animal in the absence of the liposome.

24. The formulation of claim 2 wherein the liposome comprises HSPC:Cholesterol:DSPG in a ratio of about 4:1:0.1.

25. The formulation of claim 2 wherein the liposome comprises DEPC:Cholesterol in a ratio of about 2:1.

26. The formulation of claim 2 wherein the liposome comprises DEPC:Cholesterol:DSPG in a ratio of about 2:1:0.1.

27. The formulation of claim 2 wherein the liposome comprises DOPC:Cholesterol in a ratio of about 2:1.

28. The formulation of claim 2 wherein the liposome comprises DMPC:Cholesterol:DSPG in a ratio of about 2:1:0.1.

29. The formulation of any one of claims 24-28 wherein the therapeutic agent is cisplatnin.

30. The formulation of any one of claims 24-28 wherein the therapeutic agent is amikacin or vancomycin.

31. A unit dosage form comprising a formulation of claim 1.

32. The unit dosage form of claim 31, which is formulated for parenteral administration.

33. A method for improving the efficacy of a therapeutic agent comprising encapsulating the agent in a liposome, wherein, 1) the elimination half-life of the therapeutic agent when administered to an animal as part of the formulation is at least as long as the elimination half-life of the therapeutic agent when administered to the same animal in the absence of the liposome, and wherein 2) the elimination half-life of the therapeutic agent when administered as part of the formulation is less than about 14 hours in a rat.

34. The method of claim 33 wherein the elimination half-life of the therapeutic agent when administered to an animal as part of the formulation is at least about 2-times as the elimination half-life of the therapeutic agent when administered to the same animal in the absence of the liposome,

35. A method for producing an anti-cancer effect in an animal comprising administering to the animal an effective amount of a formulation as described in claim 1 wherein the therapeutic agent is an anticancer agent.

36. A method for producing an antibiotic effect in an animal comprising administering to the animal an effective amount of a formulation as described in claim 1 wherein the therapeutic agent is an antibiotic agent.

37. A pharmaceutical composition comprising a formulation as described in claim 1 and a pharmaceutically acceptable diluent or carrier.

38. The composition of claim 37 which is formulated for parenteral administration.
